(19) United States
(12) Patent Application Publication
(10) Pub. No.: US 2025/0302340 A1
(43) Pub. Date: Oct. 2, 2025

(54) IMAGING MODULE AND WEARABLE ELECTRONIC DEVICE

(71) Applicant: FUJIFILM Corporation, Tokyo (JP)

(72) Inventors: Yukito SAITOH, Minamiashigara-shi (JP); Chie MORIYA, Minamiashigara-shi (JP)

(73) Assignee: FUJIFILM Corporation, Tokyo (JP)

(21) Appl. No.: 19/235,714

(22) Filed: Jun. 12, 2025

Related U.S. Application Data
(63) Continuation of application No. PCT/JP2023/044195, filed on Dec. 11, 2023.

(30) Foreign Application Priority Data
Dec. 22, 2022 (JP) 2022-205822
Oct. 26, 2023 (JP) 2023-183867

(51) Int. Cl. A61B 5/1171; A61B 5/00

(52) U.S. Cl. CPC A61B 5/1171; A61B 5/0077; A61B 5/681

(57) ABSTRACT

Provided is an imaging module having a biometric authentication function, which is suitable for a wearable electronic device, is thin, and can be made compact. An imaging module is included in a wearable electronic device, the imaging module including: a light emitting element that is provided to face a measurement target side in a worn state and that emits light to a measurement target; a light field camera that is provided to face the measurement target side in a worn state and that images the light emitted from the light emitting element to the measurement target; and a processor configured to process an image obtained by imaging the light emitted from the light emitting element to the measurement target using the light field camera, in which the light field camera includes a liquid crystal lens array and an image sensor array in order from the measurement target side.

### CROSS-REFERENCE TO RELATED APPLICATIONS

This application is a Continuation of PCT International Application No. PCT/JP2023/044195, filed on Dec. 11, 2023, which claims priority under 35 U.S.C. § 119(a) to Japanese Patent Application No. 2022-205822, filed on Dec. 22, 2022, and Japanese Patent Application No. 2023-183867, filed on Oct. 26, 2023. The above applications are hereby expressly incorporated by reference, in their entirety, into the present application.

### BACKGROUND OF THE INVENTION

### 1. Field of the Invention

The present invention relates to an imaging module having a biometric authentication function and a wearable electronic device.

### 2. Description of the Related Art

In recent years, a wearable electronic device is attracting attention mainly for healthcare applications. Since the wearable electronic device is always worn by a measurement subject, the wearable electronic device can easily collect, record, and manage information related to the privacy of the measurement subject, such as health information of a pulse and a body temperature, or movement information using an acceleration sensor and a global positioning system (GPS).

Further, since the wearable electronic device is integrated with the measurement subject and is always operating, it is possible to easily perform entrance management or financial settlement only by holding the wearable electronic device in a state of being worn by using electronic means such as wireless communication.

Meanwhile, a wearable electronic device closely related to such measurement subject information needs to have high security in order to protect personal information. Biometric authentication, which is excellent in convenience and confidentiality, has attracted attention as a personal authentication technology of protecting security. The biometric authentication is a technology of authenticating using human biological information.

As the biometric authentication technology in the related art, authentication using a fingerprint, an iris, a voice, a face, and a vein is known. Among these, in the biometric authentication using the vein, internal information of a body is used, so that the counterfeit resistance is excellent.

As a biometric authentication device using a vein, for example, JP2009-289287A discloses a device in which a finger position is guided to a specific position and stable collation can be performed on a captured image without the need for registration or rotation correction.

### SUMMARY OF THE INVENTION

As described above, in the method of correcting the position of the measurement target, it is required to image a wide range and to keep a distance between the measurement target and a camera to be equal to or greater than a certain distance, and thus a size of the authentication device in the related art is increased. In addition, from the viewpoint of accurately obtaining the internal information of the body, it is necessary to perform imaging with a large camera with a lens, and a thick and relatively large imaging module is required, so that there is a problem in that it is difficult to mount an imaging module having a biometric authentication function on a wearable electronic device such as a watch that is thin and needs to be made compact.

Therefore, an object of the present invention is to provide an imaging module having a biometric authentication function, which is suitable for a wearable electronic device, is thin, and can be made compact.

As a result of intensive studies to solve the above-described object, the present inventors have found that a liquid crystal lens array, which is ultra-thin and can be focused to a short focal point even though the liquid crystal lens array is a flat surface, can be applied as a substitute for a lens array included in the imaging module. That is, the present inventors have found that the above-described object can be achieved by the following configurations.
[1] An imaging module that is included in a wearable electronic device, the imaging module comprising: a light emitting element that is provided to face a measurement target side in a worn state and that emits light to a measurement target; a light field camera that is provided to face the measurement target side in a worn state and that images the light emitted from the light emitting element to the measurement target; and a processor configured to process an image obtained by imaging the light emitted from the light emitting element to the measurement target using the light field camera, in which the light field camera includes a liquid crystal lens array and an image sensor array in order from the measurement target side.
[2] The imaging module according to [1], in which the image sensor array included in the light field camera is arranged in accordance with the liquid crystal lens array, and the processor is configured to extract one feature from at least one captured image and determine whether or not the feature matches a reference feature.
[3] The imaging module according to [1] or [2], in which the imaging module has a biometric authentication function.
[4] The imaging module according to any one of [1] to [3], in which a circularly polarizing plate is disposed between the liquid crystal lens array and the measurement target side.
[5] The imaging module according to any one of [1] to [4], in which the processor is configured to determine an inclination of the light field camera and compensate for the determined inclination.

According to the aspects of the present invention, it is possible to provide the imaging module having a biometric authentication function, which is suitable for the wearable electronic device, is thin, and can be made compact.

### BRIEF DESCRIPTION OF THE DRAWINGS

FIG. 1 is a schematic perspective view showing an example of a wearable electronic device including an imaging module that embodies the present invention.

FIG. 2 is a schematic perspective view showing an example of a part of a configuration of the imaging module that embodies the present invention.

FIG. 3 is a schematic perspective view showing an example of a field of view of a liquid crystal lens that embodies the present invention.

FIG. 4 is a schematic view showing an example of a liquid crystal alignment pattern of a liquid crystal layer of the liquid crystal lens.

FIG. 5 is a schematic perspective view showing an example of a part of a configuration of another imaging module that embodies the present invention.

### DESCRIPTION OF THE PREFERRED EMBODIMENTS

Hereinafter, embodiments of the present invention will be described. In the present embodiment, an authentication method using a wrist will be described in particular, but the present invention can also be applied to a case in which an authentication subject part of a body is different, such as a palm, a finger, or an ear. It should be noted that, although the following configuration requirements are described based on representative embodiments of the present invention, the present invention is not limited to such embodiments.

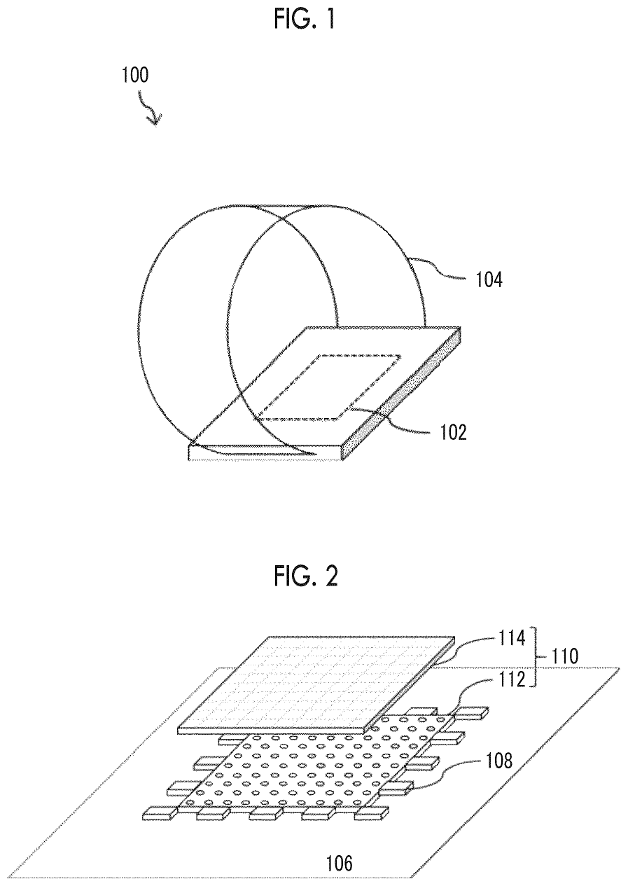

In the present embodiment, the authentication method using the wrist as a measurement target will be described in detail. A wearable electronic device and the technology described in the present specification use an imaging module including a light field camera. FIG. 1 is a schematic perspective view showing an example of the wearable electronic device including the imaging module that embodies the present invention. In FIG. 1, an imaging module 102 is configured to be located on a measurement target 106 (see FIG. 2) side of a wearable electronic device 100, but the position of the imaging module 102 is not limited to this. For example, the imaging module 102 may be located at any position in a band 104 and may be located at any position on the wearable electronic device 100. The imaging module 102 includes a light emitting element, a light field camera, and a processor. In such an imaging module 102, light, such as infrared light, penetrates deep into a measurement target part. The reflected or transmitted light is captured by the light field camera.

FIG. 2 is a schematic perspective view showing an example of a part of a configuration of the imaging module that embodies the present invention. In FIG. 2, a plurality of light emitting elements 108 are configured to be adjacent to a liquid crystal lens array, but the positions or the number of the light emitting elements 108 are not limited to this. For example, the light emitting element 108 may be located at any position in the band 104 (see FIG. 1), and may be located at any position in the wearable electronic device 100 (see FIG. 1). Further, one light emitting element 108 may be used, or a plurality of light emitting elements 108 may be used.

The light emitting element 108 is, for example, a light emitting diode (LED), may be an infrared emitter, and emits light to the measurement target 106. In addition, in a case in which the light guide element is used by being incorporated into a terminal with a built-in liquid crystal screen, such as a mobile phone, light output from the liquid crystal screen may be used as the light emitting element. In a case of control, the respective LEDs may be controlled to the same brightness, or the respective LEDs may be individually controlled.

A light field camera 110 and the light emitting element 108 are suitably a combination of a light field camera in a green wavelength range in a case of visible light or a light field camera sensitive to near-infrared light in a wavelength range of about 700 nm to 1000 nm, and a light source in a wavelength range of the light field camera. In a case in which near-infrared light is used, a filter that cuts light having a wavelength other than the near-infrared range may be mounted in the light field camera 110 to prevent extra information other than the measurement target from being reflected in a captured image.

The light field camera 110 captures not only the intensity of the received light but also the intensity of the light received by specific rays received from various directions.

The light field camera 110 includes a liquid crystal lens array 112 and an image sensor array 114 in which image sensors having fields of view defined by the respective liquid crystal lenses are arranged.

A plurality of liquid crystal lenses are arranged in the liquid crystal lens array 112.

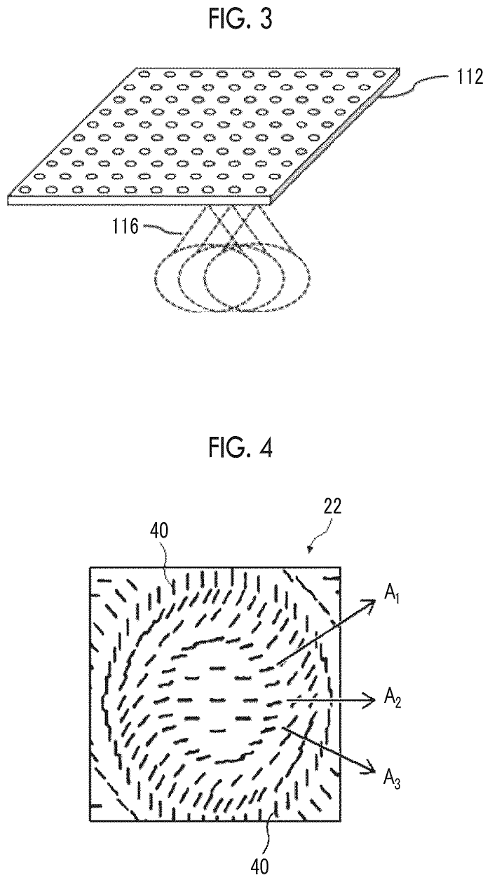

Each liquid crystal lens limits the field of view of each image sensor to the series of rays. Accordingly, each pixel value of each image sensor can be associated with the information based on a relationship of the liquid crystal lens with respect to a specific pixel of the image sensor. A resolution of the light field camera is increased with a configuration in which the fields of view of the respective liquid crystal lenses overlap each other. FIG. 3 is a schematic perspective view showing an example of a field of view 116 of the liquid crystal lens that embodies the present invention.

Regarding the liquid crystal lens, for example, WO2022/050321A1 can be referred to. Regarding the production of the liquid crystal lens array, the liquid crystal lens array may be produced with reference to WO2022/050321A1 and a plurality of lenses may be transferred onto separate substrates, or interference exposure is performed on one substrate, the substrate may be shifted in position, interference exposure may be repeated again, and then the liquid crystal layer may be formed.

FIG. 4 shows a liquid crystal alignment pattern of the liquid crystal layer of the liquid crystal lens as an example. In the liquid crystal alignment pattern in a single unit 22 of the liquid crystal lens which is a component of the liquid crystal lens array, a concentric circular pattern is provided in which one direction in which a direction of an optical axis of a liquid crystal compound 40 changes while continuously rotating to A₁, A₂, and A₃ is provided in a concentric circular shape from an inner side toward an outer side.

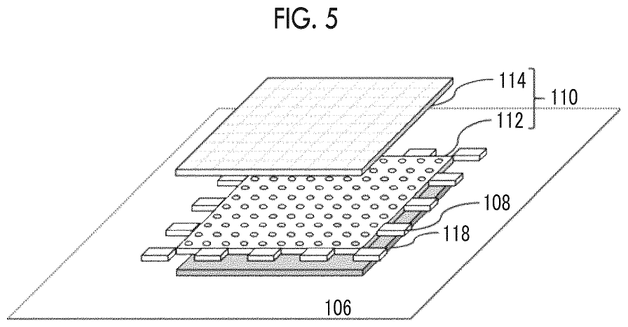

Further, a circularly polarizing plate 118 may be disposed between the liquid crystal lens array 112 and the measurement target 106. FIG. 5 is a schematic perspective view showing an example of a part of a configuration of another imaging module that embodies the present invention. By circularly polarizing the light incident to the liquid crystal lens array 112, the light after passing through the liquid crystal lens array 112 is condensed on the image sensor array 114, and thus more accurate detection can be performed.

The image sensor array 114 has a plurality of light receiving elements having sensitivity in wavelength ranges of visible light and infrared light. The image sensor array 114 has, for example, three types of complementary metal oxide semiconductors (CMOS) or charge coupled device (CCD) elements having light receiving sensitivities of blue (B), green (G), and red (R), and these are disposed in a lattice form as known as a Bayer array.

The processor is implemented as a component of any wearable electronic device capable of processing, receiving, or transmitting data or a command. The processor may be, for example, a microprocessor, a central processing unit (CPU), an application-specific integrated circuit (ASIC), a digital signal processor (DSP), or a combination of such devices. The processor in the present specification means a single processor or processing unit, a plurality of processors, a plurality of processing units, or other appropriately configured computing elements.

The processor includes, for example, a CPU, and can perform a synthetic focus operation on the image of the light field camera. In the synthetic focus operation, images of layers at distances different from a measurement target surface can be constructed by combining pixel values of different images. The synthetic focus operation is well known in the technical field, and various examples thereof are shown. Therefore, the processor can extract a feature of the measurement target from one or a plurality of layers of images constructed during the synthetic focus operation. The processor can compare the extracted feature of the measurement target with an original feature to determine a score, and determine whether or not the measurement target is authenticated in accordance with the score. A measurement subject of a watch is authenticated to allow the measurement subject to access the watch or to allow the measurement subject to access a function of the watch.

The processor may be configured to operate the light emitting element and the light field camera to acquire the image of the light field camera from the light field camera.

In addition, the imaging module 102 may be configured to compensate for an inclination of the light field camera. The inclination can be compensated by the processor related to the imaging module. An inclination sensor includes a set of proximity sensors. The proximity sensor may be a capacitive sensor, an optical sensor, another type of sensor, or a combination of these sensors. The proximity sensor can determine each distance between a surface of the imaging module and the measurement target surface.

By combining these members, it is possible to provide the imaging module having the biometric authentication function, which is thin and can be made compact.

In another embodiment, by disposing the circularly polarizing plate between the liquid crystal lens array and the measurement target side, more accurate detection can be performed.

In addition, in another embodiment, the inclinations of the imaging module and the light field camera with respect to the measurement target surface, or the inclination of the measurement target surface can be determined using the light field camera.

In still another embodiment, the processor can determine the inclinations of the imaging module and the light field camera with respect to the measurement target surface, or the inclination of the measurement target surface, report to the measurement subject that the inclination is present, prompt the measurement subject to reposition the imaging module or the wearable electronic device, and instruct the measurement subject of the method of the reposition.

### EXPLANATION OF REFERENCES

100: wearable electronic device
102: imaging module
104: band
106: measurement target
108: light emitting element
110: light field camera
112: liquid crystal lens array
114: image sensor array
116: field of view
118: circularly polarizing plate

What is claimed is:

1. An imaging module that is included in a wearable electronic device, the imaging module comprising:
a light emitting element that is provided to face a measurement target side in a worn state and that emits light to a measurement target;
a light field camera that is provided to face the measurement target side in a worn state and that images the light emitted from the light emitting element to the measurement target; and
a processor configured to process an image obtained by imaging the light emitted from the light emitting element to the measurement target using the light field camera,
wherein the light field camera includes a liquid crystal lens array and an image sensor array in order from the measurement target side.

2. The imaging module according to claim 1,
wherein the image sensor array included in the light field camera is arranged in accordance with the liquid crystal lens array, and
the processor is configured to extract one feature from at least one captured image and determine whether or not the feature matches a reference feature.

3. The imaging module according to claim 1,
wherein the imaging module has a biometric authentication function.

4. The imaging module according to claim 1,
wherein a circularly polarizing plate is disposed between the liquid crystal lens array and the measurement target side.

5. The imaging module according to claim 1,
wherein the processor is configured to determine an inclination of the light field camera and compensate for the determined inclination.

6. The imaging module according to claim 2,
wherein the imaging module has a biometric authentication function.

7. The imaging module according to claim 2,
wherein a circularly polarizing plate is disposed between the liquid crystal lens array and the measurement target side.

8. The imaging module according to claim 2,
wherein the processor is configured to determine an inclination of the light field camera and compensate for the determined inclination.

9. The imaging module according to claim 3,
wherein a circularly polarizing plate is disposed between the liquid crystal lens array and the measurement target side.

10. The imaging module according to claim 3,
wherein the processor is configured to determine an inclination of the light field camera and compensate for the determined inclination.

11. The imaging module according to claim 4,
wherein the processor is configured to determine an inclination of the light field camera and compensate for the determined inclination.
